(19) United States
(12) Patent Application Publication
(10) Pub. No.: US 2026/0064260 A1
(43) Pub. Date: Mar. 5, 2026

(54) METHOD AND SYSTEM FOR DEVELOPING HOSPITAL INFORMATION SYSTEM APPLICATION

(71) Applicant: ASUSTeK COMPUTER INC., Taipei City (TW)

(72) Inventors: Fei-Wen Cheng, Taipei City (TW); Chun-Yu Liao, Taipei City (TW); Ting-Hsuan Wang, Taipei City (TW); Chun-Ting Lin, Taipei City (TW); Shih-Wei Huang, Taipei City (TW)

(73) Assignee: ASUSTeK COMPUTER INC., Taipei City (TW)

(21) Appl. No.: 19/212,715

(22) Filed: May 20, 2025

(30) Foreign Application Priority Data
Aug. 27, 2024 (TW) 113132139

(51) Int. Cl. G06F 3/04847; G06F 3/04845; G16H 40/20

(52) U.S. Cl. CPC G06F 3/04847; G06F 3/04845; G16H 40/20

(57) ABSTRACT

Provided are a method and a system for developing hospital information system application. The method includes the following steps. A user interface frame is built, in which the user interface frame includes a plurality of user interface components. A widget is generated by using the user interface components of the user interface framework. A visual editor is configured to edit the widget in an operation page of a hospital information system application.

### CROSS-REFERENCE TO RELATED APPLICATION

This application claims the priority benefit of Taiwan application serial no. 113132139, filed on Aug. 27, 2024. The entirety of the above-mentioned patent application is hereby incorporated by reference herein and made a part of this specification.

### BACKGROUND

### Technical Field

The disclosure relates to a method and a system for developing hospital information system application.

### Description of Related Art

There are some significant problems in the development process of conventional hospital information system (HIS). The problems include a lack of modular functionality and significant duplication of code, which generally lead to several major consequences as follows. First, due to the lack of proper modular functionality, similar functions are usually implemented repeatedly in different scenarios, which leads to the significant duplication of code. For example, if different departments or hospitals have similar needs, developers need to write similar codes repeatedly, which increases the cost of system development and maintenance. In addition, customized modifications are usually implemented through hard coding. However, although the approach can quickly meet specific needs, the approach also makes the system more complex and difficult to maintain. Whenever an HIS application needs to be updated or fixed, developers have to carefully review and modify the hard-coded parts to avoid introducing new bugs or inconsistencies. In other words, when the HIS application needs to update functionality or fix bugs, the developers have to carefully review and test all related codes to ensure that the modification does not have unexpected effects on other parts. The operation is not only time-consuming and labor-intensive, but can also easily lead to version control problems and reduced software stability.

### SUMMARY

The disclosure provides a method for developing hospital information system application. The method includes the following steps. A user interface (UI) frame is built, in which the user interface frame includes multiple user interface components. A widget is generated by using the multiple user interface components of the user interface frame. A visual editor is used to edit the widget in an operation page of a hospital information system application.

The disclosure provides a system for developing hospital information system application, including a storage device and a processor. The storage device is recorded with multiple commands. The processor is coupled to the storage device and configured to perform the following operations. A user interface frame is built, in which the user interface frame includes multiple user interface components. A widget is generated by using the multiple user interface components of the user interface frame. A visual editor is used to edit the widget in an operation page of a hospital information system application.

Based on the above, in the embodiment of the disclosure, after building the user interface frame including the multiple user interface components, the user interface frame can be configured to generate the widgets corresponding to different functions. Therefore, various functions in the operation page of the hospital information system application can be implemented by the widgets. Based on the above, the functions of hospital information system application can be realized through modular widgets. Not only does the disclosure improve development efficiency and code reuse, the disclosure can also more flexibly meet the needs of different users, while reducing the cost and risk of system maintenance.

### BRIEF DESCRIPTION OF THE DRAWINGS

FIG. 1 is a block diagram of a system for developing hospital information system application according to an embodiment of the disclosure.

FIG. 2 is a flow chart of a method for developing hospital information system application according to an embodiment of the disclosure.

FIG. 3 is a schematic diagram of configuring appearance parameters of multiple user interface components according to an embodiment of the disclosure.

FIG. 4 is a schematic diagram of a widget according to an embodiment of the disclosure.

FIG. 5 is a schematic diagram of a widget in an editing operation page according to an embodiment of the disclosure.

FIG. 6A and FIG. 6B are schematic diagrams of an operation page of a hospital information system application according to an embodiment of the disclosure.

FIG. 7 is a flow chart of a method for developing hospital information system application according to an embodiment of the disclosure.

### DESCRIPTION OF THE EMBODIMENTS

Some embodiments of the disclosure will be described in detail below with reference to the accompanying drawings. The reference numerals cited in the following description will be regarded as the same or similar components when the same reference numerals appear in different drawings. The embodiments are only part of the disclosure and do not disclose all possible implementations of the disclosure. Rather, the embodiments are only examples of a system and a method within the scope of the patent application of the disclosure.

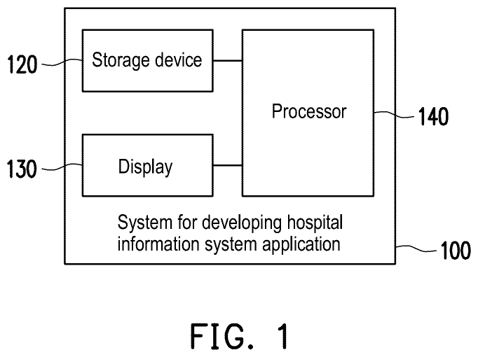

Please refer to FIG. 1. In this embodiment, a system 100 for developing hospital information system application may include a storage device 120, a display 130, and a processor 140. In some embodiments, the system 100 for developing hospital information system application may be implemented by one or more electronic devices with computing capabilities. The electronic devices may include network storage devices, servers, personal computer devices, or combinations thereof. When the system 100 for developing hospital information system application is implemented by multiple electronic devices, the electronic devices may be connected via a network or/with a wired transmission interface.

The storage device 120 is configured to store data and software modules (such as operating systems, applications, and drivers) for the processor 140 to access, and other data, and the device may be, for example, any type of fixed or removable random access memory (RAM), read-only memory (ROM), flash memory, conventional hard drive, solid state drive, or a combination thereof.

The display 130 is, for example, a liquid crystal display (LCD), a light-emitting diode (LED) display, an organic light-emitting diode display (OLED), or other types of displays, and the disclosure is not limited thereto. In this embodiment, the display 130 may display, for example, a program compilation interface, an application operation interface, and a graphical interface for visual editing.

The processor 140 is coupled to the storage device 120 and the display 130. The processor 140 is, for example, a central processing unit (CPU) or an application processor (AP), or other programmable general-purpose or special-purpose microprocessors, digital signal processors (DSP), image signal processors (ISP), graphics processing units (GPU), or other similar devices, integrated circuits, and combinations thereof. In some embodiments, the processor 140 may access and execute the software module recorded in the storage device 120 to implement a method for developing hospital information system application in an embodiment of the disclosure. The above-mentioned software modules may be broadly interpreted to mean, for example, commands, command sets, codes, programs codes, programs, applications, software packages, threads, and functions.

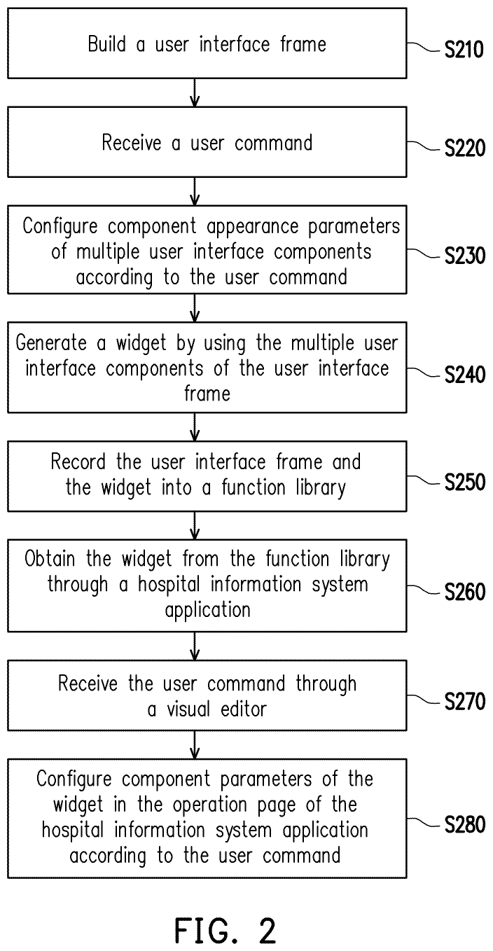

Please refer to FIG. 1 together with FIG. 2. The method of the embodiment is applicable to the system 100 for developing hospital information system application. The detailed steps of the method for developing hospital information system application of the embodiment will be described below with each component of the system 100 for developing hospital information system application.

It should be noted that the hospital information system application (HIS application) may be a front-end application, such as a web application, but is not limited thereto. The hospital information system application may provide doctors with business support such as registration of diagnostic records, operating room reservations, and patient information management. Alternatively, the hospital information system application may provide patients with business support such as online appointment, queue registration, and payment.

In Step S210, the processor 140 builds a user interface frame (UI framework), in which the user interface frame includes multiple user interface components (UI components). In other words, developers may develop multiple user interface components specifically for hospital information system applications. The multiple user interface components may include, for example, buttons, forms, select boxes, dialog boxes, navigation bars, input boxes, and text boxes. The processor 140 may use a front-end framework (such as Vue3, React, or Angular) to manage and build the user interface components to build the user interface frame including the multiple user interface components. Each user interface component may take the form of a single file component (SFC). The single file component may encapsulate the logic (such as the logic implemented by JavaScript), templates (such as the templates implemented by hypertext markup language (HTML)), and styles (such as the styles implemented by cascading style sheets (CSS)) of a single user interface component.

In Step S220, the processor 140 receives a user command. In Step S230, the processor 140 configures component appearance parameters of the multiple user interface components according to the user command. In other words, the processor 140 may change the appearance parameters of the user interface components according to the user command. The appearance parameters may include, for example, color, size, and shape. The processor 140 may receive the user command through a visual editor of the user interface frame.

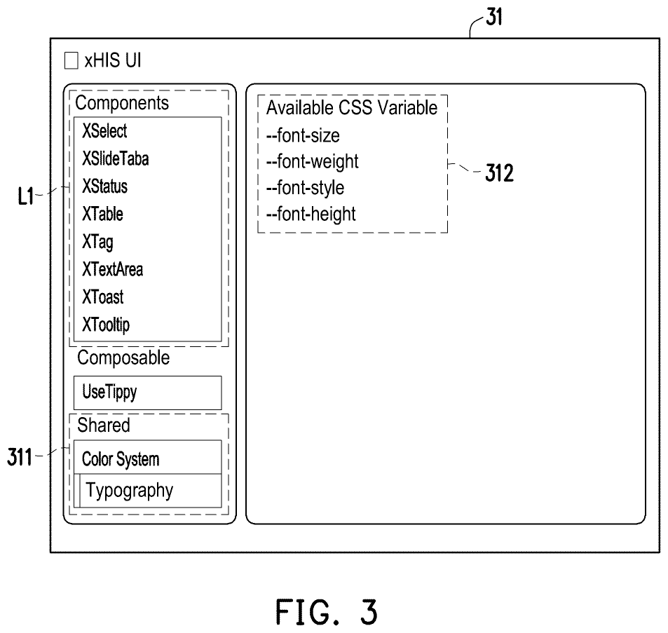

For example, please refer to FIG. 3, which is a schematic diagram of configuring the appearance parameters of the multiple user interface components according to an embodiment of the disclosure. A graphical interface 31 of the visual editor of the user interface frame includes a user interface component list L1, and the user interface component list L1 lists the multiple user interface components. In addition, the graphical interface 31 further includes a shared style setting field 311. The shared style setting field 311 includes, for example, a color system and typography. When a developer clicks on the typography in the shared style setting field 311, an available CSS variable field 312 of the graphical interface 31 may display multiple typography variables, such as a font size variable “font-size”. The developer may issue the user command to edit the typography variables. Based on above, according to the operation performed by the developer on the graphical interface 31, the processor 140 may uniformly adjust the typography and colors of the multiple user interface components, thereby improving the consistency of the user interface components. That is, when the user interface frame is utilized to implement a hospital information system application, the appearance of the same user interface component across multiple operation pages of the hospital information system application can be adjusted synchronously through quick and concise operations.

In Step S240, the processor 140 uses the multiple user interface components of the user interface frame to generate a widget. In some embodiments, the widget includes at least one of the multiple user interface components and the functional logic. The widget is a user-facing module that can be configured to display information and interact with users (such as doctors). The widgets are configured to modularize multiple functions of the hospital information system application. Different widgets are generated for different business or functional requirements. The widgets may be configured to implement specific business functions. For example, the widgets may include a personnel login function and a medication prescribing function. That is, in some embodiments, the hospital information system application is constructed based on multiple widgets. Furthermore, the processor 140 may generate the widgets based on one or more user interface components defined by the UI framework, as well as colors and typography defined by the UI framework.

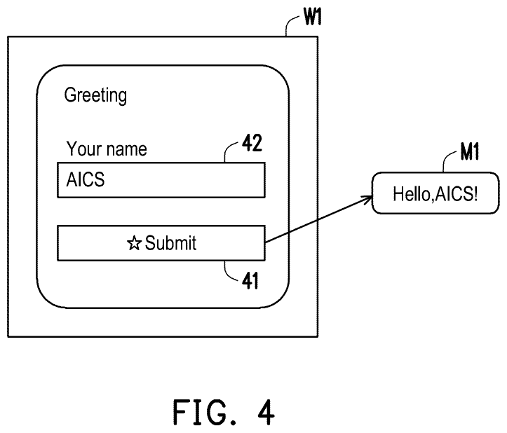

Please refer to FIG. 4, which is a schematic diagram of a widget according to an embodiment of the disclosure. The developer may implement a widget W1 based on a UI framework created by the developer. The widget W1 includes a user interface component 41 (that is, a button) and a user interface component 42 (that is, a text input box). The developer may design the structure, appearance, and functional logic of the widget W1 by themselves. For example, the processor 140 may configure the font size and decorative pattern of the user interface component 41 according to the input of the developer. The processor 140 may configure interactive behavior of the user interface component 41 according to the input of the developer. For example, in the example, when the user interface component 41 in the widget W1 of the hospital information system application is clicked, the processor 140 calls a function and display a message M1. When using the user interface component 41 and the user interface component 42 to generate the widget W1, the processor 140 needs to import the user interface component 41 and the user interface component 42 from a user interface component library. In some embodiments, the processor 140 may generate the widget W1 according to the program code input by the developer. In some embodiments, the processor 140 may generate the widget W1 based on HTML, CSS, JavaScript and a UI framework created by the developer.

In Step S250, the processor 140 records the user interface frame and the widget into a function library. In Step S260, the processor 140 obtains the widget from the function library through the hospital information system application. That is, when the developer completes the development of the user interface frame and the widget, the processor 140 may publish the user interface frame and the widget to the corresponding function library. In detail, the developer may push the program code of the user interface frame and the widget to continuous integration and continuous deployment (CICD) and automated systems for in-depth automated testing. The CICD and automated systems log the latest program code into a function library and notify the hospital information system application of the availability of the new version. Therefore, the HIS application may download the user interface frame and the widget designed by the developer.

It is worth mentioning that in an embodiment of the disclosure, the processor 140 may use a visual editor to edit the widget in the operation page of the hospital information system application. In this case, the developer, hospital information managers, or medical staff may use the visual editor to adjust the functions and page layout provided by the operation page of the hospital information system application.

In Step S270, the processor 140 receives a user command through the visual editor. When the processor 140 detects a specific user operation, the processor 140 may provide the visual editor of the operation page of the hospital information system application. The specific user operation is, for example, pressing a hot key or moving the mouse to a target location. Afterward, the developer may use the visual editor to issue user command for editing the widget.

In Step S280, the processor 140 configures component parameters of the widget in the operation page of the hospital information system application according to the user command. Therefore, through selecting the appropriate widget and configuring the component parameters of the widget, the customization of the page layout for the hospital information system application can be achieved.

Based on the above, the same widget may be applied to multiple operation pages of the hospital information system application, and the developer does not need to write the program code repeatedly. In addition, the same widget may have different appearance and display position in different operation pages.

In some embodiments, the processor 140 may configure the position and size of the widget in the operation page according to the user command. In some embodiments, the user command includes a drag and drop operation. In other words, the developer may use the drag and drop operation to modify the position and size of the widget in the operation page. For example, the processor 140 may move a certain widget from a first position to a second position in the operation page according to the drag and drop operation. Alternatively, the processor 140 may enlarge or reduce the size of a certain widget (that is, the display size in the operation page) according to the drag and drop operation.

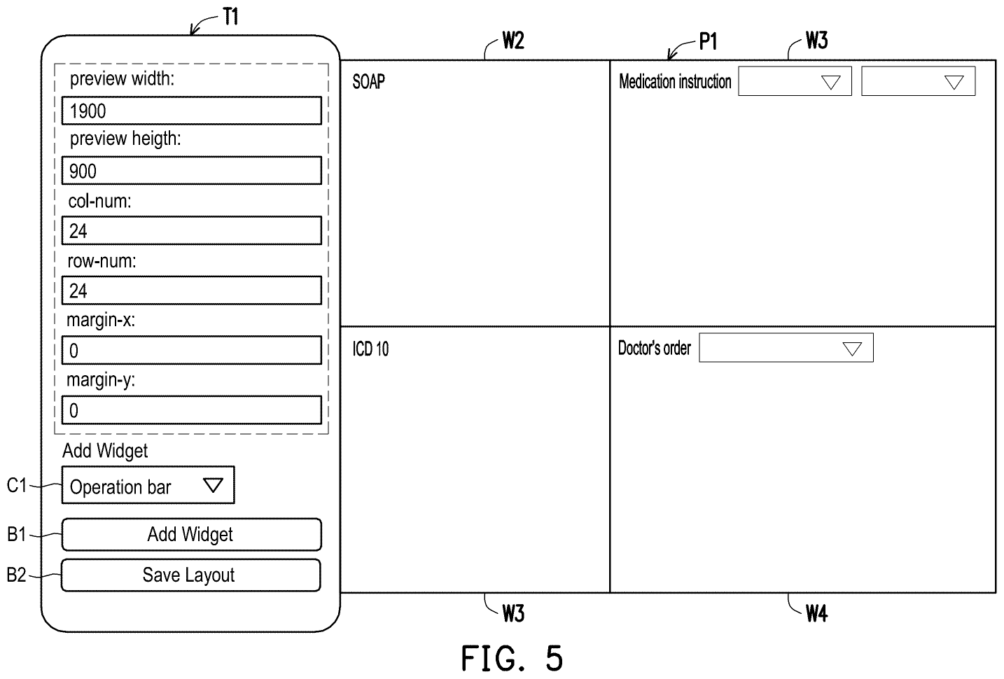

For example, please refer to FIG. 5, which is a schematic diagram of a widget in an editing operation page according to an embodiment of the disclosure. The processor 140 may provide a visual editor 51 for the operation page. The visual editor 51 includes a page editing area P1 and a setting toolbar T1. The page editing area P1 may be configured to preview the operation page and edit the operation page. The page editing area P1 displays multiple widgets W2, W3, W4, and W5 of the operation page. The processor 140 may adjust the positions and sizes of the widgets W2, W3, W4, and W5 according to the drag and drop operation applied to the page editing area P1. When the mouse moves to a specific boundary (for example, the left boundary) of the page editing area P1, the processor 140 may provide the setting toolbar T1. The setting toolbar T1 may be configured to add widgets, save page layouts, and display component parameters (such as position, size, and additional attribute) of the widgets W2, W3, W4, and W5. For example, the developer may select and add a certain widget by clicking on a selection field C1, and click on an item B1 to confirm adding the widget. The developer may click on an item B2 to save the page layout.

In some embodiments, the processor 140 may configure the additional attribute of the widget in the operation page according to the user command. The additional attribute is configured to indicate whether an additional UI behavior in the widget is enabled or disabled. Specifically, in the embodiment of the disclosure, the same widget may have different UI behaviors in different operation interfaces. For example, when the additional attribute of the widget in a first operation page indicates that the additional UI behavior is enabled (such as the additional attribute is set to ‘true’), the widget in the first operation page provides the additional UI behavior. When the additional attribute of the widget in a second operation page indicates that the additional UI behavior is disabled (such as the additional attribute is set to ‘false’), the widget in the second operation page does not provide the additional UI behavior.

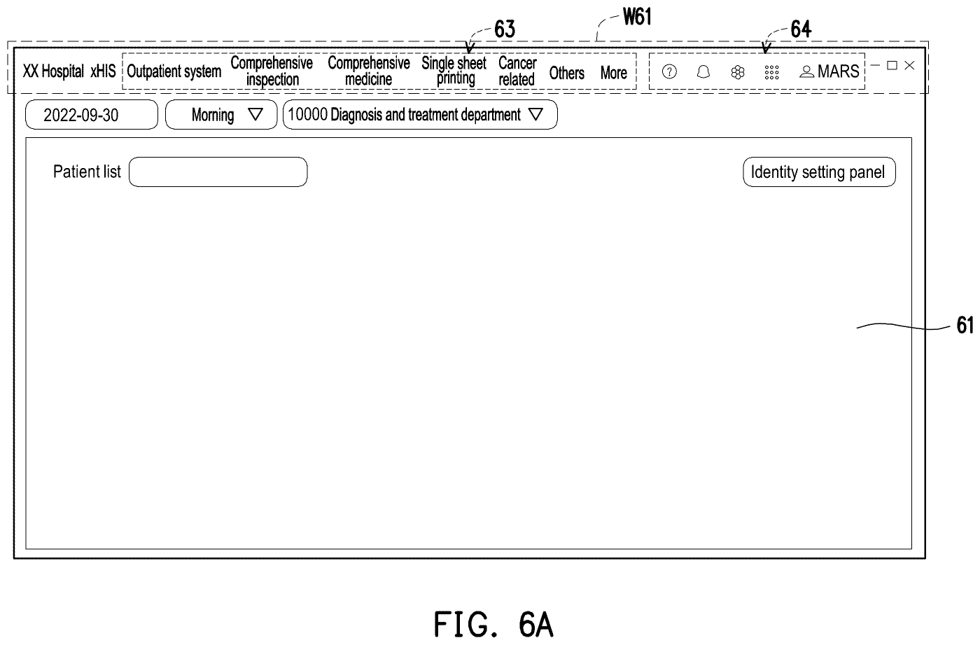

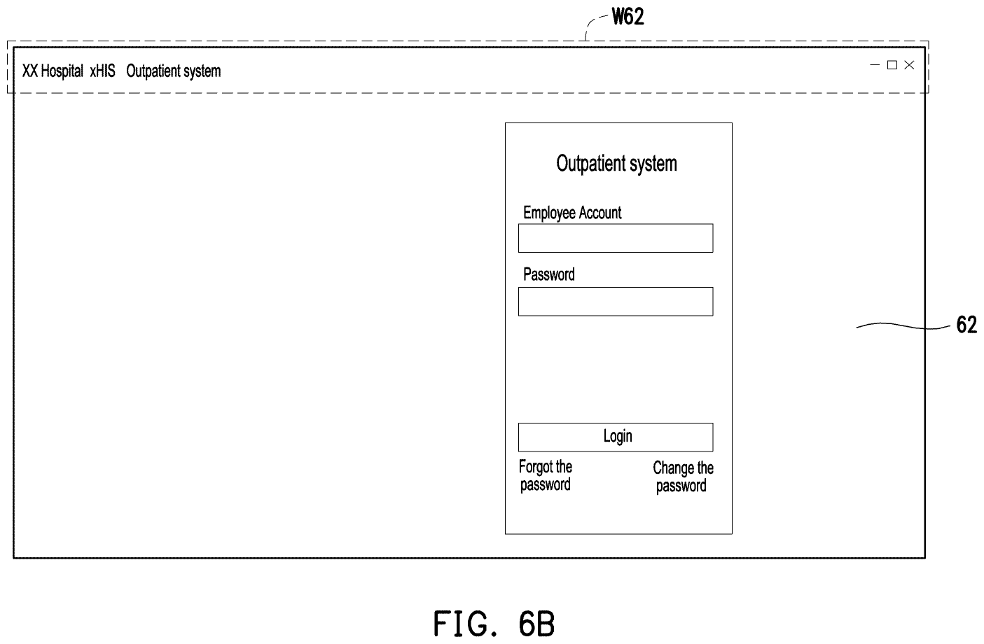

For example, please refer to FIG. 6A and FIG. 6B, which are schematic diagrams of an operation page of the hospital information system application according to an embodiment of the disclosure. Based on a first additional attribute and a second additional attribute of a widget W61 in an operation page 61 being configured as ‘true’, the first additional attribute and the second additional attribute respectively indicate that the additional UI behavior in the widget W61 is enabled. In the example of FIG. 6A, in response to the first additional attribute and the second additional attribute of the widget W61 being configured as ‘true’, the widget W61 displays a UI object 63 and a UI object 64. On the other hand, based on a first additional attribute and a second additional attribute of a widget W62 in an operation page 62 being configured as ‘false’, the first additional attribute and the second additional attribute respectively indicate that the additional UI behavior in the widget W62 is disabled. In the example of FIG. 6B, in response to the first additional attribute and the second additional attribute of the widget W62 being configured as ‘false’, the widget W62 does not display the UI objects associated with the first additional attribute and the second additional attribute. It may be seen that a single widget may provide different UI behaviors in different operation pages.

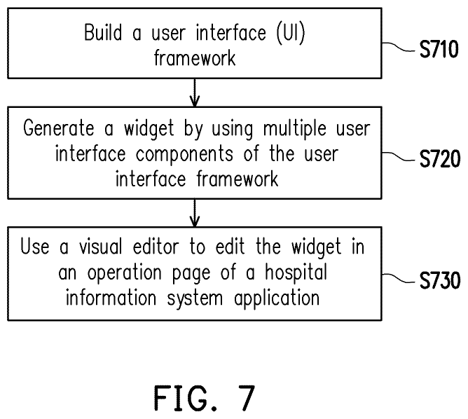

Please refer to FIG. 7, which is a flow chart of a method for developing hospital information system application according to an embodiment of the disclosure. In Step S710, a user interface frame is built, in which the user interface frame includes the multiple user interface components. In Step S720, a widget is generated by using the multiple user interface components of the user interface framework. In Step S730, a visual editor is configured to edit the widget in an operation page of a hospital information system application. However, each step in FIG. 7 has been described in detail above, so details will not be repeated here. In addition, the method in FIG. 7 may be used together with the above example embodiments or may be used separately, and the disclosure is not limited thereto.

In summary, in the embodiments of the disclosure, after building the user interface frame including the multiple user interface components, widgets corresponding to different functions can be generated by using the user interface frame. Therefore, various functions in the operation page of the hospital information system application may be implemented by the widgets. Based on the above, the disclosure is helpful to improve development efficiency and ease of maintenance. The method of the disclosure emphasizes modularity, reusability, and user-centered design, enabling the HIS application to flexibly meet the changing needs of medical providers. In addition, by using the visual editor to edit the widget on the operation page, a WYSIWYG effect can be achieved, so that the page layout of the HIS application can meet user needs.

Although the disclosure has been disclosed in the above embodiments, the embodiments are not intended to limit the disclosure. Persons with ordinary knowledge in the relevant technical field may make some changes and modifications without departing from the spirit and scope of the disclosure. Therefore, the protection scope of the disclosure shall be determined by the appended claims and the equivalent scope thereof.

What is claimed is:

1. A method for developing hospital information system application, comprising:
building a user interface (UI) frame, wherein the user interface frame comprises a plurality of user interface components;
generating a widget by using the plurality of user interface components of the user interface frame; and
using a visual editor to edit the widget in an operation page of a hospital information system application.

2. The method for developing hospital information system application as claimed in claim 1, further comprising:
recording the user interface frame and the widget to a function library; and
obtaining the widget from the function library through the hospital information system application.

3. The method for developing hospital information system application as claimed in claim 1, wherein using the visual editor to edit the widget in the operation page of the hospital information system application comprises:
receiving a user command through the visual editor; and
configuring component parameters of the widget in the operation page of the hospital information system application according to the user command.

4. The method for developing hospital information system application as claimed in claim 3, wherein configuring the component parameters of the widget in the operation page of the hospital information system application according to the user command further comprises:
configuring a position and a size of the widget in the operation page according to the user command.

5. The method for developing hospital information system application as claimed in claim 4, wherein the user command comprises a drag and drop operation.

6. The method for developing hospital information system application as claimed in claim 3, wherein configuring the component parameters of the widget in the operation page of the hospital information system application according to the user command further comprises:
configuring an additional attribute of the widget in the operation page according to the user command, wherein the additional attribute indicates whether an additional UI behavior in the widget is enabled or disabled.

7. The method for developing hospital information system application as claimed in claim 3, wherein the visual editor comprises a page editing area and a setting toolbar.

8. The method for developing hospital information system application as claimed in claim 1, wherein the widget comprises at least one of the plurality of user interface components and a functional logic.

9. The method for developing hospital information system application as claimed in claim 1, further comprising:
receiving a user command; and
configuring component appearance parameters of the plurality of user interface components according to the user command.

10. A system for developing hospital information system application, comprising:
a storage device recorded with a plurality of commands; and
a processor connected to the storage device and configured to execute the commands to:
build a user interface (UI) frame, wherein the user interface frame comprises a plurality of user interface components;
generate a widget by using the plurality of user interface components of the user interface frame; and
use a visual editor to edit the widget in an operation page of a hospital information system application.
